(19) United States
(12) Patent Application Publication
(10) Pub. No.: US 2024/0271194 A1
(43) Pub. Date: Aug. 15, 2024

(54) RATIOMETRIC SYMBOLS AND SEQUENTIAL CODING FOR MULTIPLEXED FISH

(71) Applicant: CALIFORNIA INSTITUTE OF TECHNOLOGY, Pasadena, CA (US)

(72) Inventors: Long CAI, Pasadena, CA (US); Chee Huat (Linus) ENG, Pasadena, CA (US); Yujing YANG, Pasadena, CA (US)

(21) Appl. No.: 18/568,005

(22) PCT Filed: Jun. 8, 2022
(86) PCT No.: PCT/US2022/032736

§ 371 Date: Dec. 7, 2023

Related U.S. Application Data
(60) Provisional application No. 63/208,364, filed on Jun. 8, 2021.

(51) Int. Cl. C12Q 1/6841; G16B 30/10

(52) U.S. Cl. CPC C12Q 1/6841; G16B 30/10; C12Q 2600/166

(57) ABSTRACT

Disclosed herein are methods for generating an ratiometric symbol for sequential hybridization barcoding for multiplexed Fluorescence In Situ Hybridization (FISH). Also, the disclosure sets forth methods, in addition to using the same, and other solutions to problems in the relevant field.

### CROSS-REFERENCE TO RELATED APPLICATIONS

This application claims the benefit of U.S. Provisional Patent Application No. 63/208,364, filed Jun. 8, 2021. The contents of the above-referenced application are hereby incorporated by reference in their entirety.

### BACKGROUND

Sequential fluorescence in situ hybridization (seqFISH) methods have been used to multiplex a large number of molecules in cells and samples. One of the major limiting factors in the experiments is imaging time, which is controlled by the number of rounds of hybridizations. On the one hand, more rounds of hybridizations applied judiciously allow dense targets to be diluted and super-resolved. On the other hand, more rounds of hybridization cost imaging time and reduce the amount of sample that can be processed. However, there may be scenarios where speed of acquisition is more important, and fewer rounds of hybridization need to be implemented to increase sample processing throughput.

### SUMMARY

The present disclosure provides methods for drastically reducing the number of rounds of hybridization needed for multiplexed Fluorescence In Situ Hybridization (FISH). This disclosure sets forth methods, in addition to using the same, and other solutions to problems in the relevant field.

In some embodiments, there is provided a method for barcoding one or more molecular targets with ratiometric symbols, comprising the steps of contacting a sample comprising a plurality of molecular targets with a first plurality of one or more primary probes, wherein the one or more primary probes interact with one or more molecular targets, and wherein each primary probe comprises one or more binding sites for a detectably labelled probe. In some embodiments, the method comprises contacting the one or more primary probes with one or more sets of ratiometric detectably labelled probes. In some embodiments, the ratiometric detectably labelled probes comprise at least a first detectably labelled probe that interacts with a first primary probe binding site. In some embodiments, the one or more sets of ratiometric detectably labelled probes comprises a second detectably labelled probe that interacts with the first primary probe binding site. In some embodiments, the label of the first detectably labelled probe is different from the label of the second detectably labelled probe. In some embodiments, the first detectably labelled probe and second detectably labelled probe contact the first primary probe binding site at a pre-determined ratio. In some embodiments, the method comprises for each set of ratiometric detectably labelled probes, imaging the intensities of the different detectably labels between different channels to determine a distinct ratio, so that the interaction of the detectably labelled probes with their primary probes is detected. In some embodiments, the method comprises generating a ratiometric symbol for each ratio. In some embodiments, the method comprises generating a non-ratiometric symbol for each molecular target. In some embodiments, the method comprises optionally repeating any of the previous embodiments, each time with one or more sets of detectably labelled probes, so that one or more molecular targets in the sample are described by a barcode, wherein at least one barcode comprises at least one ratiometric symbol, and wherein at least one molecular target can be differentiated from another molecular target in the sample by a difference in their barcodes.

In some embodiments, there is provided a method for barcoding one or more molecular targets with ratiometric symbols, comprising the steps of contacting a sample comprising a plurality of molecular targets with a first plurality of one or more primary probes, wherein the one or more primary probes interact with one or more molecular targets, and wherein each primary probe comprises one or more amplifier sequences. In some embodiments, the method comprises contacting the one or more primary probes with one or more amplifiers to form one or more amplification scaffolds, wherein the amplifiers comprise one or more amplifier sequences, and wherein the amplifiers sequences comprise one or more adaptor sequences. In some embodiments, the method comprises contacting the one or more amplifier scaffolds, with one or more sets of ratiometric adaptor probes. In some embodiments, each set of the ratiometric adaptor probes comprises at least a first adaptor probe that interacts with a first adaptor sequence on the amplifier scaffold. In some embodiments, each set of the ratiometric probes comprises a second detectably labelled probe that interacts with a second ratiometric adaptor probe. In some embodiments, the first adaptor probe and second adaptor probe contact the first primary probe binding site at a pre-determined ratio. In some embodiments, the method comprises contacting the one or more sets of ratiometric adaptor probes with one or more sets of detectably labelled probes. In some embodiments, each set of the detectably labelled probes comprises at least a first detectably labelled probe that interacts with a first ratiometric adaptor probe. In some embodiments, each set of the detectably labelled probes comprises a second detectably labelled probe that interacts with a second ratiometric adaptor probe. In some embodiments, the label of the first detectably labelled probe is different from the label of the second detectably labelled probe. In some embodiments, the method comprises for each set of ratiometric adaptor probes, imaging the intensities of the different detectably labels between different channels to determine a distinct ratio, so that the interaction of the adaptor probes with their primary probes is detected. In some embodiments, the method comprises generating a ratiometric symbol for each ratio. In some embodiments, the method comprises optionally repeating steps any of the previous embodiments, each time with one or more sets of ratiometric adaptor probes so that one or more molecular targets in the sample are described by a barcode, wherein at least one barcode comprises at least one ratiometric symbol, and wherein at least one molecular target can be differentiated from another molecular target in the sample by a difference in their barcodes.

In some embodiments, the methods are used to reduce the number of rounds of hybridization needed for multiplexed Fluorescence In Situ Hybridization (FISH). In contrast to other methods, that require a primary probe to have a specific number of binding sites for each detectably labelled probe, the methods described herein makes use of a competitive interaction in a set of ratiometric detectably labelled probes to barcode a molecular target that is swift and efficient.

As an example, a molecular target (RNA-1) in a cell can be targeted by primary probes containing an RNA-1 specific binding sequence as well as a sequence that can be bound by a detectably labeled probe. The detectably labelled probe can be labeled with either Cy3 or AF750N. By mixing the Cy3 and the AF750N labeled probes at a fixed ratio (e.g. 0:5, 1:4, 2:3, 3:2, 4:1, 5:0), 6 distinct ratios can be distinguished from each dot in the image, allowing 6 molecular species to be uniquely identified. Thus, 6 ratiometric symbols can be generated from the ratio of Cy3 and AF750N signals on a RNA target.

Additionally, if the primary probes have additional binding sites for another detectably labelled probe with a different fluorophore, then another six symbols can be generated. Overall 6×6=36 barcodes can be generated to uniquely identify 36 different RNA species using 4 color channels in only 1 round of hybridization. If the primary probes have additional binding sites for other detectably labelled probes, the method scales exponentially in additional rounds of hybridization and allows A^{N} total distinguishable barcodes, where A is the size of the symbol and N is the number of binding sites. For example, 6⁶=46,656 barcodes allow the entire transcriptome of 24,000 genes to be coded in only 3 rounds of hybridization with 4 fluorescent channels. In contrast, without the ratiometric symbols, coding for 24,000 genes requires at least 8 rounds of hybridization, 4⁸=65,536, more than 2.6 times longer in terms of hybridization and imaging time. Therefore, the methods described herein should allow an improvement in efficiency.

In some embodiments, the methods are used to reduce the number of rounds of hybridization needed for linked amplification tethered with exponential radiance (LANTERN) (LANTERN). LANTERN provides strong fluorescent signals to generate ratiometric symbols via the competitive binding of adaptors or detectably labelled probes interacting with adaptors probes. Ratiometric symbols can be generated more accurately on amplified signals from the primary probes because there are a larger number of binding sites and less random noise in binding, which scales as VS, where S is the number of binding sites.

The methods described herein should be more accurate than ratiometric coding directly on the primary probe, wherein the primary probe comprises a specific number of sites for the detectably labelled probe to interact with. This is because the interaction of a primary probe to a molecular target in the cell may be highly random, resulting in ratiometric symbols that are less and are more difficult to distinguish from each other. In addition, competitive binding of readout probes should be easier to implement than having different number of binding sites on the primary probe. This is because coding for larger ratio differences (such as 8:1) requires, in the case of oligonucleotides, longer primary probes sequences, which add costs to the probes and increases nonspecific binding. A competitive binding implementation should have an advantage that requires only a single readout site on the primary probe and a ratio that can be flexibly adjusted by changing the relative concentration of the readout probes, enabling quick adjustments of the ratiometric symbols.

### BRIEF DESCRIPTIONS OF THE DRAWINGS

FIG. 1. An example of implementing ratiometric symbols. RNAs are targeted by primary probes, which are amplified through either padlock or rolling circle mechanisms. Competitive binding of secondary probes with different concentrations on the amplicons generate the ratiometric symbols on each RNA molecule.

FIG. 2. Images of 6 ratiometric levels are shown for the same sample of cells. Two channels (Cy3 and AF750N) are shown for each ratiometric level. The intensity ratios are 0:5, 1:4, 2:3, 3:2, 4:1, 5:0. Each dot corresponds to a single RNA molecule and is present in two fluorescent channels at a defined intensity ratio.

FIG. 3. Histogram of the dot intensity ratios for different symbols in cells. 6 distinct ratiometric symbols are designed with Cy3 labeled and the AF750N labeled secondary probes mixed at a fixed ratio (0:5, 1:4, 2:3, 3:2, 4:1, 5:0). 6 distinct histograms were computed from the single molecule intensities from the sample.

FIG. 4. Barcoding schemes for 36 targets with ratiometric symbols. (A). Amplified DNA scaffold from LANTERN encodes binding sites for two competing detectably labelled probes at designated concentrations with distinct fluorophores. (B) Barcoding scheme for 36 targets generated from 6×6 ratiometric symbols. Each ratiometric symbol has its unique amplifier sequences associated with the designated ratio. (C) Orthogonal detectably labelled probes are imaged in their respective wavelength via a fluorescence microscopy. The fluorescence intensity after calibration between the fluorescence channels are used to calculate the ratio, forming the ratiometric symbols used in the codebook. Bottom panel demonstrates the distinct ratio generated from 36 unique amplifier sequences to encode for 36 targets, in this case, mRNAs from 36 different genes.

FIG. 5. Representative raw images from ratiometric barcoding experiments. 36 barcodes are demonstrated via a 6×6 ratiometric barcoding scheme. The detectably labelled oligonucleotides comprise fluorescent dyes Cy3b, AF750N (for the left detectably labeled probes) and Alexa 488 and Alexa 647 (for right detectably labelled probes). The smaller sized panels are zoomed images of the yellow boxes (AF: Alexa Fluor).

FIG. 6. Ratiometric barcoding in single cells. (A) A 2D histogram calculating the left and right side ratios of all detected dots from the experiment shown in FIG. 5. (B) A 2D heatmap demonstrating that 10 distinct ratiometric symbols could be generated by using the concentration ratio of 0:9, 1:8, 2:7, 3:6, 4:5, 5:4, 6:3, 7:2, 8:1, 9:0 for both left and right side adaptors.

FIG. 7. Encoding with more amplifier sequences on primary probes expands the encoding capacity dramatically. For example, since each amplifier sequence can provide 36 barcodes in theory by using 6×6 ratio-metric scheme or 100 barcodes using 10×10 ratio-metric scheme, inclusion of two unique amplifier sequences can then provide 6×6×6×6=1296 barcodes or 10×10×10×10=10,000 barcodes in 2 rounds of sequential hybridizations. Additional amplifier sequences can be incorporated in the primary probe such that 2 or more amplified scaffold can form on the target molecule. For example, three amplifiers would provide 6×6×6×6×6×6=46656 barcodes in 3 rounds of sequential hybridization.

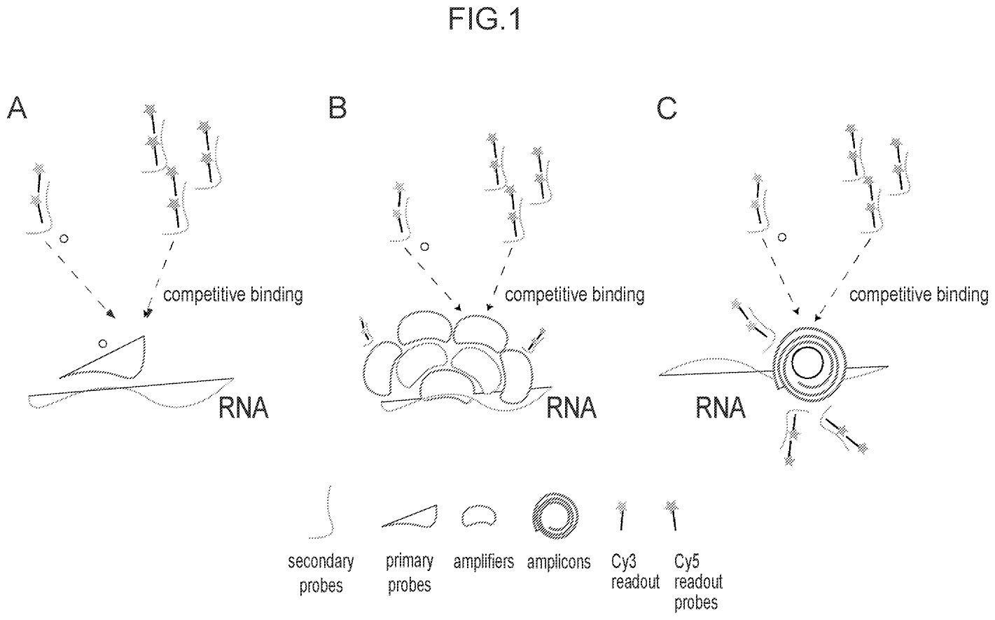

### DETAILED DESCRIPTION

The following description is presented to enable one of ordinary skill in the art to make and use the disclosed subject matter and to incorporate it in the context of applications. Various modifications, as well as a variety of uses in different applications, will be readily apparent to those skilled in the art, and the general principles defined herein may be applied to a wide range of embodiments. Thus, the present disclosure is not intended to be limited to the embodiments presented, but is to be accorded the widest scope consistent with the principles and novel features disclosed herein.

### Definitions

Unless otherwise noted, terms are to be understood according to conventional usage by those of ordinary skill in the relevant art.

As used herein, the terms “approximately” or “about” in reference to a number are generally taken to include numbers that fall within a range of 5%, 10%, 15%, or 20% in either direction (greater than or less than) of the number unless otherwise stated or otherwise evident from the context (except where such number would be less than 0% or exceed 100% of a possible value).

The term “oligonucleotide” refers to a polymer or oligomer of nucleotide monomers, containing any combination of nucleobases, modified nucleobases, sugars, modified sugars, phosphate bridges, or modified bridges.

Oligonucleotides can be of various lengths. In particular embodiments, oligonucleotides can range from about 2 to about 200 nucleotides in length. In various related embodiments, oligonucleotides, single-stranded, double-stranded, and triple-stranded, can range in length from about 4 to about 10 nucleotides, from about 10 to about 50 nucleotides, from about 20 to about 50 nucleotides, from about 15 to about 30 nucleotides, from about 20 to about 30 nucleotides in length. In some embodiments, the oligonucleotide is from about 9 to about 39 nucleotides in length. In some embodiments, the oligonucleotide is at least 4 nucleotides in length. In some embodiments, the oligonucleotide is at least 5 nucleotides in length. In some embodiments, the oligonucleotide is at least 6 nucleotides in length. In some embodiments, the oligonucleotide is at least 7 nucleotides in length. In some embodiments, the oligonucleotide is at least 8 nucleotides in length. In some embodiments, the oligonucleotide is at least 9 nucleotides in length. In some embodiments, the oligonucleotide is at least 10 nucleotides in length. In some embodiments, the oligonucleotide is at least 11 nucleotides in length. In some embodiments, the oligonucleotide is at least 12 nucleotides in length. In some embodiments, the oligonucleotide is at least 15 nucleotides in length. In some embodiments, the oligonucleotide is at least 20 nucleotides in length. In some embodiments, the oligonucleotide is at least 25 nucleotides in length. In some embodiments, the oligonucleotide is at least 30 nucleotides in length. In some embodiments, the oligonucleotide is a duplex of complementary strands of at least 18 nucleotides in length. In some embodiments, the oligonucleotide is a duplex of complementary strands of at least 21 nucleotides in length.

As used herein, the term “probe” or “probes” refers to any molecules, synthetic or naturally occurring, that can attach themselves directly or indirectly to a molecular target (e.g., an mRNA sample, DNA molecules, protein molecules, RNA and DNA isoform molecules, single nucleotide polymorphism molecules, and etc.). For example, a probe can include a nucleic acid molecule, an oligonucleotide, a protein (e.g., an antibody or an antigen binding sequence), or combinations thereof. For example, a protein probe may be connected with one or more nucleic acid molecules to for a probe that is a chimera. As disclosed herein, in some embodiments, a probe itself can produce a detectable signal. In some embodiments, a probe is connected, directly or indirectly via an intermediate molecule, with a signal moiety (e.g., a dye or fluorophore) that can produce a detectable signal.

As used herein, the term “sample” refers to a biological sample obtained or derived from a source of interest, as described herein. In some embodiments, a source of interest comprises an organism, such as an animal or human. In some embodiments, a biological sample comprises biological tissue or fluid. In some embodiments, a biological sample is or comprises bone marrow; blood; blood cells; ascites; tissue or fine needle biopsy samples; cell-containing body fluids; free floating nucleic acids; sputum; saliva; urine; cerebrospinal fluid, peritoneal fluid; pleural fluid; feces; lymph; gynecological fluids; skin swabs; vaginal swabs; oral swabs; nasal swabs; washings or lavages such as a ductal lavages or broncheoalveolar lavages; aspirates; scrapings; bone marrow specimens; tissue biopsy specimens; surgical specimens; feces, other body fluids, secretions, and/or excretions; and/or cells therefrom, etc. In some embodiments, a biological sample is or comprises cells obtained from an individual. In some embodiments, a sample is a “primary sample” obtained directly from a source of interest by any appropriate means. For example, in some embodiments, a primary biological sample is obtained by methods selected from the group consisting of biopsy (e.g., fine needle aspiration or tissue biopsy), surgery, collection of body fluid (e.g., blood, lymph, feces etc.), etc. In some embodiments, as will be clear from context, the term “sample” refers to a preparation that is obtained by processing (e.g., by removing one or more components of and/or by adding one or more agents to) a primary sample. For example, filtering using a semi-permeable membrane. Such a “processed sample” may comprise, for example nucleic acids or proteins extracted from a sample or obtained by subjecting a primary sample to techniques such as amplification or reverse transcription of mRNA, isolation and/or purification of certain components, etc. In some embodiments, the term “sample” refers to a nucleic acid such as DNA, RNA, transcripts, or chromosomes. In some embodiments, the term “sample” refers to nucleic acid that has been extracted from the cell.

As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and/or chemical phenomena.

As disclosed herein, the term “label” generally refers to a molecule that can recognize and bind to specific target sites within a molecular target in a cell. For example, a label can comprise an oligonucleotide that can bind to a molecular target in a cell. The oligonucleotide can be linked to a moiety that has affinity for the molecular target. The oligonucleotide can be linked to a first moiety that is capable of covalently linking to the molecular target. In certain embodiments, the molecular target comprises a second moiety capable of forming the covalent linkage with the label. In particular embodiments, a label comprises a nucleic acid sequence that is capable of providing identification of the cell which comprises or comprised the molecular target. In certain embodiments, a plurality of cells is labelled, wherein each cell of the plurality has a unique label relative to the other labelled cells.

As disclosed herein, the term “barcode” generally refers to a symbol sequence of a label produced by methods described herein. The barcode sequence typically is of a sufficient length and uniqueness to identify a molecular target.

In some embodiments, the targets are selected from transcripts, RNA, DNA loci, chromosomes, DNA, protein, lipids, glycans, cellular targets, organelles, and any combinations thereof. In certain embodiments, the transcripts, RNA, DNA loci, chromosomes, DNA, protein, lipids, glycans, cellular targets, organelles, and any combinations thereof are conjugated to an oligonucleotide.

### Overview

The present disclosure provides methods for drastically reducing the number of rounds of hybridization needed for multiplexed Fluorescence In Situ Hybridization (FISH). This disclosure sets forth methods, in addition to using the same, and other solutions to problems in the relevant field.

In some embodiments, there is provided a method for barcoding one or more molecular targets with ratiometric symbols, comprising the steps of contacting a sample comprising a plurality of molecular targets with a first plurality of one or more primary probes, wherein the one or more primary probes interact with one or more molecular targets, and wherein each primary probe comprises one or more binding sites for a detectably labelled probe. In some embodiments, the method comprises contacting the one or more primary probes with one or more sets of ratiometric detectably labelled probes. In some embodiments, the ratiometric detectably labelled probes comprise at least a first detectably labelled probe that interacts with a first primary probe binding site. In some embodiments, the one or more sets of ratiometric detectably labelled probes comprise a second detectably labelled probe that interacts with the first primary probe binding site. In some embodiments, the label of the first detectably labelled probe is different from the label of the second detectably labelled probe. In some embodiments, the first detectably labelled probe and second detectably labelled probe contact the first primary probe binding site at a pre-determined ratio. In some embodiments, the method comprises for each set of ratiometric detectably labelled probes, imaging the intensities of the different detectably labels between different channels to determine a distinct ratio, so that the interaction of the detectably labelled probes with their primary probes is detected. In some embodiments, the method comprises generating a ratiometric symbol for each ratio. In some embodiments, the method comprises generating a non-ratiometric symbol for each molecular target. In some embodiments, the method comprises generating a non-ratiometric symbol for each molecular target, so that one or more molecular targets in the sample are described by a barcode, wherein at least one barcode comprises at least one ratiometric symbol, and wherein at least one molecular target can be differentiated from another molecular target in the sample by a difference in their barcodes. In some embodiments, the method comprises optionally repeating any of the previous steps, each time with one or more sets of detectably labelled probes, so that one or more molecular targets in the sample are described by a barcode, wherein at least one barcode comprises at least one ratiometric symbol, and wherein at least one molecular target can be differentiated from another molecular target in the sample by a difference in their barcodes.

In some embodiments, there is provided a method for barcoding one or more molecular targets with ratiometric symbols, comprising the steps of contacting a sample comprising a plurality of molecular targets with a first plurality of one or more primary probes, wherein the one or more primary probes interact with one or more molecular targets, and wherein each primary probe comprises one or more amplifier sequences. In some embodiments, the method comprises contacting the one or more primary probes with one or more amplifiers to form one or more amplification scaffolds, wherein the amplifiers comprise one or more amplifier sequences, and wherein the amplifiers sequences comprise one or more adaptor sequences. In some embodiments, the method comprises contacting the one or more amplifier scaffolds, with one or more sets of ratiometric adaptor probes. In some embodiments, each set of the ratiometric adaptor probes comprise at least a first adaptor probe that interacts with a first adaptor sequence on the amplifier scaffold. In some embodiments, each set of the ratiometric probes comprises a second detectably labelled probe that interacts with a second ratiometric adaptor probe. In some embodiments, the first adaptor probe and second adaptor probe contact the first primary probe binding site at a pre-determined ratio. In some embodiments, the method comprises contacting the one or more sets of ratiometric adaptor probes with one or more sets of detectably labelled probes. In some embodiments, each set of the detectably labelled probes comprises at least a first detectably labelled probe that interacts with a first ratiometric adaptor probe. In some embodiments, each set of the detectably labelled probes comprises a second detectably labelled probe that interacts with a second ratiometric adaptor probe. In some embodiments, the label of the first detectably labelled probe is different from the label of the second detectably labelled probe. In some embodiments, the method comprises for each set of ratiometric adaptor probes, imaging the intensities of the different detectably labels between different channels to determine a distinct ratio, so that the interaction of the adaptor probes with their primary probes is detected. In some embodiments, the method comprises generating a ratiometric symbol for each ratio. In some embodiments, the method comprises generating a ratiometric symbol for each ratio, so that one or more molecular targets in the sample are described by a barcode, wherein at least one barcode comprises at least one ratiometric symbol, and wherein at least one molecular target can be differentiated from another molecular target in the sample by a difference in their barcodes. In some embodiments, the method comprises optionally repeating any of the previous embodiments, each time with one or more sets of ratiometric adaptor probes, so that one or more molecular targets in the sample are described by a barcode, wherein at least one barcode comprises at least one ratiometric symbol, and wherein at least one molecular target can be differentiated from another molecular target in the sample by a difference in their barcodes.

In some embodiments, the method of any of the previous embodiments comprises a third detectably labelled probe that interacts with a first primary probe binding site. In some embodiments, the third detectably labelled probe is different from the label of the first or second detectably labelled probe. In some embodiments, the first detectably labelled probe, second detectably labelled probe, and third detectably labelled probe contact the first primary probe binding site at a pre-determined ratio.

In some embodiments, the methods of any of the previous embodiments comprises a fourth detectably labelled probe that interacts with a first primary probe binding site. In some embodiments, the fourth detectably labelled probe is different from the label of the first, second, or third detectably labelled probe. In some embodiments, the first detectably labelled probe, second detectably labelled probe, and third detectably labelled probe contact the first primary probe binding site at a pre-determined ratio.

In some embodiments, the method of any of the previous embodiments comprises the steps of contacting a sample comprising a plurality of molecular targets with a first plurality of detectably labelled probes comprising at least a first detectably labelled probe that interacts with a first molecular target. In some embodiments, first plurality of detectably labelled probes comprises a second detectably labelled probe that interacts with a second molecular target. In some embodiments, the first detectably labelled probe is different from the second detectably labelled probe. In some embodiments, the method comprises imaging the sample after the first contacting step so that interaction of the detectably labelled probes with their target nucleic acids are detected. In some embodiments, the method comprises generating a non-ratiometric symbol for each molecular target. In some embodiments, the method comprises repeating the contacting and imaging steps, each time with a new plurality of detectably labelled probes, so that a molecular target in the sample is described by a barcode, wherein at least one barcode comprises at least one non-ratiometric symbol, and wherein the barcode can be differentiated from another target nucleic acid in the sample by a difference in their barcodes.

In some embodiments, the method of any of the previous embodiments comprises contacting the sample with a plurality of non-ratiometric probes before contacting the sample with a plurality of ratiometric probes. In some embodiments, the method of any of the previous embodiments comprises contacting the sample with a plurality of non-ratiometric probes at the same time as contacting the sample with a plurality of ratiometric probes. In some embodiments, the method of any of the previous embodiments comprises contacting the sample with a plurality of non-ratiometric probes after contacting the sample with a plurality of ratiometric probes.

In some embodiments, the method of any of the previous embodiments comprises contacting the one or more primary probes with 2, 3, 4, 5, 6, 7, or 8 sets of ratiometric detectably labelled probes.

In some embodiments, the method of any of the previous embodiments comprises contacting the one or more amplifier scaffolds with 2, 3, 4, 5, 6, 7, or 8 sets of ratiometric adaptor probes.

In some embodiments, the method of any of the previous embodiments comprises amplifying the primary probes by rolling circle, padlock, branched DNA, ClampFISH, LANTERN, or any combination thereof before step (ii).

### Sample and Molecular Targets

In some embodiments, the method comprises analyzing samples, wherein the samples comprise bacterial cells, archaeal cells, eukaryotic cells, or a combination thereof. In certain embodiments, the samples comprise tissues, cells, or extracts from cells. In certain embodiments, the samples comprise cells obtained from patients. In certain embodiments, the samples comprise fluids obtained from patients.

In some embodiments, the sample comprises molecular targets that are selected from proteins, modified proteins, transcripts, RNA, DNA loci, exogenous proteins, exogenous nucleic acids, hormones, carbohydrates, small molecules, biologically active molecules, and combinations thereof. In some embodiments, the targets comprise subcellular features.

### Primary Probes

In some embodiments, the method comprises contacting a sample comprising a plurality of molecular targets with a plurality of one or more primary probes.

In some embodiments, the primary probe is selected from proteins, modified proteins, RNA, oligonucleotides, antibodies, antibody fragments, and combinations thereof.

In some embodiments, the primary probe comprises an oligonucleotide. In some embodiments, the detectably labelled probe comprises an oligonucleotide with a detectably moiety.

In some embodiments, the primary probe comprises oligonucleotides that are at least 5 nucleotides long. In some embodiments, the primary probe comprises oligonucleotides that are at least 6 nucleotides long. In some embodiments, the primary probe comprises oligonucleotides that are at least 7 nucleotides long. In some embodiments, the primary probe comprises oligonucleotides that are at least 8 nucleotides long. In some embodiments, the primary probe comprises oligonucleotides that are at least 9 nucleotides long. In some embodiments, the primary probe comprises oligonucleotides that are at least 10 nucleotides long. In some embodiments, the primary probe comprises oligonucleotides that are at least 11 nucleotides long. In some embodiments, the primary probe comprises oligonucleotides that are at least 12 nucleotides long. In some embodiments, the primary probe comprises oligonucleotides that are at least 13 nucleotides long. In some embodiments, the primary probe comprises oligonucleotides that are at least 14 nucleotides long. In some embodiments, the primary probe comprises oligonucleotides that are at least 15 nucleotides long. In some embodiments, the primary probe comprises oligonucleotides that are at least 16 nucleotides long. In some embodiments, the primary probe comprises oligonucleotides that are at least 17 nucleotides long. In some embodiments, the primary probe comprises oligonucleotides that are at least 18 nucleotides long. In some embodiments, the primary probe comprises oligonucleotides that are at least 19 nucleotides long. In some embodiments, the primary probe comprises oligonucleotides that are at least 20 nucleotides long. In some embodiments, the primary probe comprises oligonucleotides that are at least 21 nucleotides long. In some embodiments, the primary probe comprises oligonucleotides that are at least 22 nucleotides long. In some embodiments, the primary probe comprises oligonucleotides that are at least 23 nucleotides long. In some embodiments, the primary probe comprises oligonucleotides that are at least 24 nucleotides long. In some embodiments, the primary probe comprises oligonucleotides that are at least 25 nucleotides long. In some embodiments, the primary probe comprises oligonucleotides that are at least 26 nucleotides long. In some embodiments, the primary probe comprises oligonucleotides that are at least 27 nucleotides long. In some embodiments, the primary probe comprises oligonucleotides that are at least 28 nucleotides long. In some embodiments, the primary probe comprises oligonucleotides that are at least 29 nucleotides long. In some embodiments, the primary probe comprises oligonucleotides that are at least 30 nucleotides long. In some embodiments, the primary probes of any of the previous embodiments comprises oligonucleotides that are less than 35, 40, 45, 50, 100 nucleotides in length.

In some embodiments, the primary probe comprises a sequence that is complementary to the molecular target. In some embodiments the sequence complementarity comprises at least 60%, 65%, 70%, 75%, 80%, 85%, 90%, 91%, 92%, 93%, 94%, 95%, 96%, 97%, 98%, 99%, or 100%.

In some embodiments, the primary probe comprises one or more amplifier sequences. In some embodiments, the primary probe comprises two or more amplifier sequences. In some embodiments, the primary probe comprises three or more amplifier sequences. In some embodiments, the primary probe comprises four or more amplifier sequences. In some embodiments, the primary probe comprises five or more amplifier sequences. In some embodiments, the primary probe comprises six or more amplifier sequences. In some embodiments, the primary probe comprises seven or more amplifier sequences. In some embodiments, the primary probe comprises eight or more amplifier sequences.

In some embodiments, the one or more amplifier sequences are the same sequences. In some embodiments, at least one of the amplifier sequences in the one or more amplifier sequences are the same. In some embodiments, the one or more amplifier sequences are different from each other. In some embodiments, at least one of the amplifier sequences in the one or more amplifier sequences are different.

In some embodiments, the amplifier sequence comprises a nucleotide sequence that is at least 5 nucleotides long. In some embodiments, the amplifier sequence comprises a nucleotide sequence that is at least 6 nucleotides long. In some embodiments, the amplifier sequence comprises a nucleotide sequence that is at least 7 nucleotides long. In some embodiments, the amplifier sequence comprises a nucleotide sequence that is at least 8 nucleotides long. In some embodiments, the amplifier sequence comprises a nucleotide sequence that is at least 9 nucleotides long. In some embodiments, the amplifier sequence comprises a nucleotide sequence that is at least 10 nucleotides long. In some embodiments, the amplifier sequence comprises a nucleotide sequence that is at least 11 nucleotides long. In some embodiments, the amplifier sequence comprises a nucleotide sequence that is at least 12 nucleotides long. In some embodiments, the amplifier sequence comprises a nucleotide sequence that is at least 13 nucleotides long. In some embodiments, the amplifier sequence comprises a nucleotide sequence that is at least 14 nucleotides long. In some embodiments, the amplifier sequence comprises a nucleotide sequence that is at least 15 nucleotides long. In some embodiments, the amplifier sequence comprises a nucleotide sequence that is at least 16 nucleotides long. In some embodiments, the amplifier sequence comprises a nucleotide sequence that is at least 17 nucleotides long. In some embodiments, the amplifier sequence comprises a nucleotide sequence that is at least 18 nucleotides long. In some embodiments, the amplifier sequence comprises a nucleotide sequence that is at least 19 nucleotides long. In some embodiments, the amplifier sequence comprises a nucleotide sequence that is at least 20 nucleotides long. In some embodiments, the amplifier sequence comprises a nucleotide sequence that is at least 21 nucleotides long. In some embodiments, the amplifier sequence comprises a nucleotide sequence that is at least 22 nucleotides long. In some embodiments, the amplifier sequence comprises a nucleotide sequence that is at least 23 nucleotides long. In some embodiments, the amplifier sequence comprises a nucleotide sequence that is at least 24 nucleotides long. In some embodiments, the amplifier sequence comprises a nucleotide sequence that is at least 25 nucleotides long. In some embodiments, the amplifier sequence comprises a nucleotide sequence that is at least 26 nucleotides long. In some embodiments, the amplifier sequence comprises a nucleotide sequence that is at least 27 nucleotides long. In some embodiments, the amplifier sequence comprises a nucleotide sequence that is less than 35, 40, 45, 50, 100 nucleotides in length.

### Amplifiers

In some embodiments, the method comprises contacting the one or more primary probes with one or more amplifiers to form one or more amplification scaffolds. In some embodiments, the amplifiers comprise one or more amplifier sequences. In some embodiments, the amplifiers sequences comprise one or more adaptor sequences.

In some embodiments, the one or more amplification scaffolds are the same. In some embodiments, the one or more amplification scaffolds are different.

In some embodiments, the amplifier sequences comprise 1, 2, 3, 4, 5, 6, 7, 8, 9, or 10 adaptor sequences.

In some embodiments, the amplifier sequences comprise one or more adaptor sequences. In some embodiments, the amplifier sequences comprise two or more adaptor sequences. In some embodiments, the amplifier sequences comprise three or more adaptor sequences. In some embodiments, the amplifier sequences comprise four or more adaptor sequences. In some embodiments, the amplifier sequences comprise five or more adaptor sequences. In some embodiments, the amplifier sequences comprise six or more adaptor sequences. In some embodiments, the amplifier sequences comprise seven or more adaptor sequences. In some embodiments, the amplifier sequences comprise eight or more adaptor sequences. In some embodiments, the amplifier sequences comprise less than 10, 15, 20, or 25 adaptor sequences.

In some embodiments, the adaptor sequences at least 5 nucleotides long. In some embodiments, the adaptor sequences at least 6 nucleotides long. In some embodiments, the adaptor sequences at least 7 nucleotides long. In some embodiments, the adaptor sequences at least 8 nucleotides long. In some embodiments, the adaptor sequences at least 9 nucleotides long. In some embodiments, the adaptor sequences at least 10 nucleotides long. In some embodiments, the adaptor sequences at least 11 nucleotides long. In some embodiments, the adaptor sequences at least 12 nucleotides long. In some embodiments, the adaptor sequences at least 13 nucleotides long. In some embodiments, the adaptor sequences at least 14 nucleotides long. In some embodiments, the adaptor sequences at least 15 nucleotides long. In some embodiments, the adaptor sequences at least 16 nucleotides long. In some embodiments, the adaptor sequences at least 17 nucleotides long. In some embodiments, the adaptor sequences at least 18 nucleotides long. In some embodiments, the adaptor sequences at least 19 nucleotides long. In some embodiments, the adaptor sequences at least 20 nucleotides long. In some embodiments, the adaptor sequences at least 21 nucleotides long. In some embodiments, the adaptor sequences at least 22 nucleotides long. In some embodiments, the adaptor sequences at least 23 nucleotides long. In some embodiments, the adaptor sequences at least 24 nucleotides long. In some embodiments, the adaptor sequences at least 25 nucleotides long. In some embodiments, the adaptor sequences at least 26 nucleotides long. In some embodiments, the adaptor sequences at least 27 nucleotides long. In some embodiments, the adaptor sequences at least 28 nucleotides long. In some embodiments, the adaptor sequences at least 29 nucleotides long. In some embodiments, the adaptor sequences at least 30 nucleotides long. In some embodiments, the adaptor sequences of any of the previous embodiments comprises nucleotides sequences that are less than 35, 40, 45, 50, 100 nucleotides in length.

### Detectably Labelled Probes

In some embodiments, the method comprises barcoding molecular targets by using detectably labelled probes.

In some embodiments, the detectably labelled probe is selected from proteins, modified proteins, RNA, oligonucleotides, antibodies, antibody fragments, and combinations thereof. In some embodiments, the detectably labelled probe further comprises a detectably moiety. In certain embodiments, the detectably moiety is a fluorophore.

In some embodiments, the detectably labelled probe comprises an oligonucleotide with a detectably moiety.

In some embodiments, the detectably labelled probe comprises oligonucleotides that are at least 5 nucleotides long. In some embodiments, the detectably labelled probe comprises oligonucleotides that are at least 6 nucleotides long. In some embodiments, the detectably labelled probe comprises oligonucleotides that are at least 7 nucleotides long. In some embodiments, the detectably labelled probe comprises oligonucleotides that are at least 8 nucleotides long. In some embodiments, the detectably labelled probe comprises oligonucleotides that are at least 9 nucleotides long. In some embodiments, the detectably labelled probe comprises oligonucleotides that are at least 10 nucleotides long. In some embodiments, the detectably labelled probe comprises oligonucleotides that are at least 11 nucleotides long. In some embodiments, the detectably labelled probe comprises oligonucleotides that are at least 12 nucleotides long. In some embodiments, the detectably labelled probe comprises oligonucleotides that are at least 13 nucleotides long. In some embodiments, the detectably labelled probe comprises oligonucleotides that are at least 14 nucleotides long. In some embodiments, the detectably labelled probe comprises oligonucleotides that are at least 15 nucleotides long. In some embodiments, the detectably labelled probe comprises oligonucleotides that are at least 16 nucleotides long. In some embodiments, the detectably labelled probe comprises oligonucleotides that are at least 17 nucleotides long. In some embodiments, the detectably labelled probe comprises oligonucleotides that are at least 18 nucleotides long. In some embodiments, the detectably labelled probe comprises oligonucleotides that are at least 19 nucleotides long. In some embodiments, the detectably labelled probe comprises oligonucleotides that are at least 20 nucleotides long. In some embodiments, the detectably labelled probe comprises oligonucleotides that are at least 21 nucleotides long. In some embodiments, the detectably labelled probe comprises oligonucleotides that are at least 22 nucleotides long. In some embodiments, the detectably labelled probe comprises oligonucleotides that are at least 23 nucleotides long. In some embodiments, the detectably labelled probe comprises oligonucleotides that are at least 24 nucleotides long. In some embodiments, the detectably labelled probe comprises oligonucleotides that are at least 25 nucleotides long. In some embodiments, the detectably labelled probe comprises oligonucleotides that are at least 26 nucleotides long. In some embodiments, the detectably labelled probe comprises oligonucleotides that are at least 27 nucleotides long. In some embodiments, the detectably labelled probe comprises oligonucleotides that are at least 28 nucleotides long. In some embodiments, the detectably labelled probe comprises oligonucleotides that are at least 29 nucleotides long. In some embodiments, the detectably labelled probe comprises oligonucleotides that are at least 30 nucleotides long. In some embodiments, the detectably labelled probes of any of the previous embodiments comprises oligonucleotides that are less than 35, 40, 45, 50, 100 nucleotides in length.

In some embodiments, the detectably labelled probe comprises a sequence that is complementary to the primary probe. In some embodiments, the sequence complementarity comprises at least 60%, 65%, 70%, 75%, 80%, 85%, 90%, 91%, 92%, 93%, 94%, 95%, 96%, 97%, 98%, 99%, or 100%.

In some embodiments, the detectably labelled probes comprise oligonucleotides with the same sequence. In some embodiments, the detectably labelled probes comprise oligonucleotides with different sequences

### Intermediate Probes

In some embodiments, the method comprises detectably labelled probes interacting with their primary probes or amplified scaffold through one or more intermediate probes. In some embodiments, the intermediate probes are adaptor probes.

In some embodiments, a detectably labeled probe interacts with its target through binding or hybridization to one or more intermediate probe. In some embodiments, the intermediate probe comprises an oligonucleotide, antibody, antibody fragment, protein, or any combination thereof.

In some embodiments, the intermediate probe binds, hybridizes, or otherwise links to the target. In some embodiments, the method comprises a detectably labeled oligonucleotide interacting with a target through hybridization with an intermediate probe hybridized to a target, wherein the intermediate probe comprises a sequence complimentary to the target, and an overhang sequence. In some embodiments, the sequence complementarity comprises at least 60%, 65%, 70%, 75%, 80%, 85%, 90%, 91%, 92%, 93%, 94%, 95%, 96%, 97%, 98%, 99%, or 100%.

In some embodiments, the intermediate probe comprises an oligonucleotide that is at least 5 nucleotides long. In some embodiments, the intermediate probe comprises an oligonucleotide that is at least 6 nucleotides long. In some embodiments, the intermediate probe comprises an oligonucleotide that is at least 7 nucleotides long. In some embodiments, the intermediate probe comprises an oligonucleotide that is at least 8 nucleotides long. In some embodiments, the intermediate probe comprises an oligonucleotide that is at least 9 nucleotides long. In some embodiments, the intermediate probe comprises an oligonucleotide that is at least 10 nucleotides long. In some embodiments, the intermediate probe comprises an oligonucleotide that is at least 11 nucleotides long. In some embodiments, the intermediate probe comprises an oligonucleotide that is at least 12 nucleotides long. In some embodiments, the intermediate probe comprises an oligonucleotide that is at least 13 nucleotides long. In some embodiments, the intermediate probe comprises an oligonucleotide that is at least 14 nucleotides long. In some embodiments, the intermediate probe comprises an oligonucleotide that is at least 15 nucleotides long. In some embodiments, the intermediate probe comprises an oligonucleotide that is at least 16 nucleotides long. In some embodiments, the intermediate probe comprises an oligonucleotide that is at least 17 nucleotides long. In some embodiments, the intermediate probe comprises an oligonucleotide that is at least 18 nucleotides long. In some embodiments, the intermediate probe comprises an oligonucleotide that is at least 19 nucleotides long. In some embodiments, the intermediate probe comprises an oligonucleotide that is at least 20 nucleotides long. In some embodiments, the intermediate probe comprises an oligonucleotide that is at least 21 nucleotides long. In some embodiments, the intermediate probe comprises an oligonucleotide that is at least 22 nucleotides long. In some embodiments, the intermediate probe comprises an oligonucleotide that is at least 23 nucleotides long. In some embodiments, the intermediate probe comprises an oligonucleotide that is at least 24 nucleotides long. In some embodiments, the intermediate probe comprises an oligonucleotide that is at least 25 nucleotides long. In some embodiments, the intermediate probe comprises an oligonucleotide that is at least 26 nucleotides long. In some embodiments, the intermediate probe comprises an oligonucleotide that is at least 27 nucleotides long. In some embodiments, the intermediate probe comprises an oligonucleotide that is at least 28 nucleotides long. In some embodiments, the intermediate probe comprises an oligonucleotide that is at least 29 nucleotides long. In some embodiments, the intermediate probe comprises an oligonucleotide that is at least 30 nucleotides long. In some embodiments, the intermediate probes of any of the previous embodiments comprises oligonucleotides that are less than 35, 40, 45, 50, 100 nucleotides in length.

In some embodiments, the intermediate probe comprises an overhang sequence that is complementary to a detectably labelled probe. In some embodiments, the sequence complementarity comprises at least 60%, 65%, 70%, 75%, 80%, 85%, 90%, 91%, 92%, 93%, 94%, 95%, 96%, 97%, 98%, 99%, or 100%.

In some embodiments, the intermediate probe comprises an overhang sequence is complementary to a bridge probe. In some embodiments, the bridge probe comprises a sequence complementary to the detectably labeled probe. In some embodiments, the bridge probe comprises a sequence complementary to an intermediate probe.

In some embodiments, the method comprises intermediate probes that are preserved through multiple contacting and imaging steps. In some embodiments, the method comprises a removing step that removes detectably labeled probes, optionally keeping the intermediate probes intact. In some embodiments, the method comprises a removing step that removes the detectably labeled probes and keeps the intermediate probes intact. In some embodiments, detectably labeled probes differ from the intermediate probes in a chemical or enzymatic perspective, so that detectably labeled oligonucleotides can be selectively removed.

In some embodiments, the method comprises an adaptor probe that is an intermediate probe. In certain embodiments, the adaptor probes are selected from proteins, modified proteins, RNA, oligonucleotides, antibodies, antibody fragments, and combinations thereof. In certain embodiments, the adaptor probes are oligonucleotides. In certain embodiments, the adaptor probe hybridizes to an amplifier scaffold. In certain embodiments, each adaptor probe comprises a sequence complementary to the primary probe and an overhang sequence. In certain embodiments, the overhang sequence is complementary to a detectably labelled probe. In certain embodiments, the overhang sequence is complementary to a bridge probe. In certain embodiments, the bridge probe is complementary to a detectably labelled probe and to an adaptor probe.

### Ratios

In some embodiments, the detectably labelled probes contact the primary probe binding site at a pre-determined ratio. In some embodiments, the method comprises generating ratiometric symbols by using different concentrations of detectably labeled probes to compete directly or indirectly for the binding sites on the primary probe at a pre-determined ratio. In certain embodiments, the detectably labelled probes compete indirectly for the binding sites on the primary probe at a predetermined ratio by adding a decoy probe to the ratio. In some embodiments, the method comprises contacting the sample with different concentrations of detectably labelled probes, wherein the different concentrations are different ratios of detectably labelled probes.

In some embodiments, each pre-determined ratio between any two detectably labelled probes is greater to or equal to 0.0. In some embodiments, each pre-determined ratio between any two detectably labelled probes is less than or equal to 1.0. In some embodiments, each pre-determined ratio between any two detectably labelled probes is between about 0.0 to 1.0. In some embodiments, each pre-determined ratio between any two of three detectably labelled probes is between about 0.0 to 1.0. In some embodiments, each pre-determined ratio between any two of four or more three detectably labelled probes is between about 0.0 to 1.0. In some embodiments, each pre-determined ratio between any two of five, six, seven, or eight or more detectably labelled probes is between about 0.0 to 1.0.

In some embodiments, each pre-determined ratio between any two detectably labelled probes is about 0.1, 0.2, 0.25, 0.3, 0.4, 0.5, 0.6, 0.7, 0.75, 0.8, 0.9, or 1.0. In some embodiments, each pre-determined ratio between any three probes is 0.10:0.20:0.70; 0.25:0.25:0.50; 0.25:0.50:0.25; 0.50:0.25:0.25; or 0.70:0.20:0.10. In some embodiments, each pre-determined ratio between any four probes is 0.10:0.10:0.10:0.70; 0.10:0.20:0.20:0.50; 0.25:0.25:0.25:0.25; 0.50:0.20:0.20:0.10; 0.70:0.10:0.10:0.10.

In some embodiments, the method comprises contacting the one or more amplifier scaffolds, with one or more sets of ratiometric adaptor probes at a pre-determined ratio. In some embodiments, the method comprises generating the ratiometric symbols by using different concentrations of ratiometric adaptor probes to compete directly or indirectly for the binding sites on the amplifier scaffolds. In certain embodiments, the ratiometric adaptor probes compete indirectly for the binding sites on the amplifier scaffolds by adding a decoy probe in a ratio. In some embodiments, the method comprises contacting the sample with different concentrations of ratiometric adaptor probes, wherein different concentrations are different ratios if adaptor probes.

In some embodiments, each pre-determined ratio between any two ratiometric adaptor probes is greater to or equal to 0.0. In some embodiments, each pre-determined ratio between any two ratiometric adaptor probes is less than or equal to 1.0. In some embodiments, each pre-determined ratio between any two ratiometric adaptor probes is between about 0.0 to 1.0. In some embodiments, each pre-determined ratio between any two of three ratiometric adaptor probes is between about 0.0 to 1.0. In some embodiments, each pre-determined ratio between any two of four or more ratiometric adaptor probes is between about 0.0 to 1.0. In some embodiments, each pre-determined ratio between any two of five, six, seven, eight, or more ratiometric adaptor probes is between about 0.0 to 1.0. In some embodiments, each pre-determined ratio between any two ratiometric adaptor probes is about 0.1, 0.2, 0.25, 0.3, 0.4, 0.5, 0.6, 0.7, 0.75, 0.8, 0.9, or 1.0. In some embodiments, each pre-determined ratio between any three ratiometric adaptor probes is 0.10:0.20:0.70; 0.25:0.25:0.50; 0.25:0.50:0.25; 0.50:0.25:0.25; or 0.70:0.20:0.10. In some embodiments, each pre-determined ratio between any four ratiometric adaptor probes is 0.10:0.10:0.10:0.70; 0.10:0.20:0.20:0.50; 0.25:0.25:0.25:0.25; 0.50:0.20:0.20:0.10; 0.70:0.10:0.10:0.10.

### Barcoding the Targets

In some embodiments, the method comprises barcoding one or more molecular targets. In some embodiments, the molecular targets that are selected from proteins, modified proteins, transcripts, RNA, DNA loci, exogenous proteins, exogenous nucleic acids, hormones, carbohydrates, small molecules, biologically active molecules, and combinations thereof. In some embodiments, the targets comprise subcellular features. For example, the nuclear lamin can be labeled with one set of barcodes, and nucleolus can be targeted with another set of barcodes. Then each sample can be uniquely labeled with a combination of barcodes on different subcellular compartments. In some embodiments, the method comprises barcoding targets, wherein the targets are different.

In some embodiments, the method comprises fluorescence detection. In some embodiments, the method comprises fluorescence detection or other methods of detection. In some embodiments, the method comprises sequential hybridization to detect target analytes.

In some embodiments, the probes are used in a method to barcode one or more molecular targets. See, for example, International PCT Patent Application No. PCT/US2014/036258, filed Apr. 30, 2014 and titled MULTIPLEX LABELING OF MOLECULES BY SEQUENTIAL HYBRIDIZATION BARCODING, the entire contents of which are herein incorporated by reference in its entirety for all purposes.

In some embodiments, the probes are used in a method for linked amplification tethered with exponential radiance (LANTERN). See, for example, International Patent Application No. PCT/US2022/021826, FILED Mar. 24, 2022, and titled LINKED AMPLIFICATION TETHERED WITH EXPONENTIAL RADIANCE, the entire contents of which are herein incorporated by reference in its entirety for all purposes.

In some embodiments, the probes are used in ClampFISH experiments. See, for example, ClampFISH detects individual nucleic acid molecules using click chemistry-based amplification, Rouhanifard S. H. et al., Nature Biotechnology 37: 84-89 (2019), the entire contents of which are herein incorporated by reference in its entirety for all purposes.

In some embodiments, the method comprises detectably labelled probes that are selected from proteins, modified proteins, RNA, oligonucleotides, antibodies, antibody fragments, and combinations thereof.

In some embodiments, the method comprises contacting each sample in the one or more samples with a first plurality of detectably labelled probes, so that the probes interact with one or more targets. In some embodiments, the method comprises imaging the sample after the first contacting step so that interaction of the detectably labelled probes with their targets is detected.

In some embodiments, the method comprises a contacting step that differs from another contacting step in the labelling of at least one of the targets.

In some embodiments, the method comprises a contacting step wherein each detectably labeled probe in the first plurality of probes is labelled with a detectably moiety.

In some embodiments, the method comprises a contacting step wherein each detectably labelled probe comprises a detectable moiety and at least one contacting step differs from another contacting step by having a different detectable moiety for each target.

In some embodiments, the method comprises a contacting step wherein at least two different detectably labelled probes that interact with a first target and wherein at least two different detectably labelled probes interact with a second target.

In some embodiments, the detectably labelled probes comprise labels selected from two, three, or four different labels.

In some embodiments, the barcode for the target in the sample comprises a signal that is amplified. In certain embodiments, the barcode for the target in a sample comprises a signal that is amplified by rolling circle, padlock, branched DNA, ClampFISH, LANTERN, or any combination thereof.

In some embodiments, the method comprises using detectably labelled probes wherein each detectably labeled probe comprises the same detectable moiety and the same sequence.

In some embodiments, the method comprises detectably labelled probes wherein each detectably labelled probes interacts with its target through one or more intermediate probes each of which is hybridized to the target.

In some embodiments, the method comprises repeating the contacting and imaging steps, each time with a new plurality of detectably labelled probes so that a target in the sample is described by a barcode, and can be differentiated from another target in the sample by a difference in their barcodes.

In some embodiments, the barcode of any of the previous embodiments comprises ratiometric, non-ratiometric symbols, and any combination thereof. In some embodiments, the barcode comprises 1, 2, 3, 4, 5, 6, 7, 8, 9, or 10 ratiometric symbols. In some embodiments, the barcode comprises 0, 1, 2, 3, 4, 5, 6, 7, 8, 9, or 10 non-ratiometric symbols.

In some embodiments, the ratiometric symbols are generated across rounds of hybridization. For example, a Cy3 detectably labelled probe may be used in the first round of hybridization and Cy5 detectably labelled probe may be used in the second round of hybridization to generate a ratiometric symbol.

In some embodiments, the method comprises an error correction round. See, for example, International Patent Application No. PCT/US2017/044994, FILED Aug. 1, 2017, and titled SEQUENTIAL PROBING OF MOLECULAR TARGETS BASED ON PSEUDO-COLOR BARCODES WITH EMBEDDED ERROR CORRECTION MECHANISM, the entire contents of which are herein incorporated by reference in its entirety for all purposes.

In some embodiments, the method comprises an error correction round performed by selecting from block codes such as Hamming codes, Reed-Solomon codes, Golay codes, or any combination thereof.

In some embodiments, the method of any of the previous embodiments further comprises an error correction step. In certain embodiments, the error correction step comprises performing additional rounds of contacting and imaging prior or in between or after steps (i)-(v).

In some embodiments, the method of any of the previous embodiments comprises assigning of ratiometric symbols comprises applying a machine learning algorithm.

### Removing Probes

In some embodiments, the method comprises a step of removing the detectably labelled probes after one or more imaging steps. In some embodiments, the step of removing the detectably labelled probes comprises contacting the plurality of detectably labelled probes with an enzyme that digests a detectably labelled probes. In some embodiments, the step of removing comprises contacting the plurality of detectably labelled probes with a DNase, contacting the plurality of detectably labelled probes with an RNase, photobleaching, strand displacement, formamide wash, heat denaturation, or combinations thereof. In some embodiments, the step of removing comprises photobleaching to remove the detectably labelled probes.

In some embodiments, the method comprises removing detectably labelled probes by using stripping reagents, wash buffers, photobleaching, chemical bleaching, and any combinations thereof.

In some embodiments, the method comprises clearing the sample. In some embodiments the sample is cleared by CLARITY.

Certain techniques for removing probes are known in the art. See, for example, International PCT Patent Application No. PCT/US2014/036258, filed Apr. 30, 2014 and titled MULTIPLEX LABELING OF MOLECULES BY SEQUENTIAL HYBRIDIZATION BARCODING, the entire contents of which are herein incorporated by reference in its entirety for all purposes.

### Imaging the Sample

In some embodiments, the method comprises imaging the detectably labelled probes. In some embodiments, the method comprises imaging the barcodes. As understood by a person having ordinary skill in the art, different technologies can be used for the imaging steps.

In some embodiments, the imaging methods comprise but are not limited to epi-fluorescence microscopy, confocal microscopy, the different types of super-resolution microscopy (PALM/STORM, SSIM/GSD/STED), and light sheet microscopy (SPIM and etc.).

In some embodiments, the imaging methods comprise exemplary super resolution technologies include, but are not limited to I⁵M and 4Pi-microscopy, Stimulated Emission Depletion microscopy (STEDM), Ground State Depletion microscopy (GSDM), Spatially Structured Illumination microscopy (SSIM), Photo-Activated Localization Microscopy (PALM), Reversible Saturable Optically Linear Fluorescent Transition (RESOLFT), Total Internal Reflection Fluorescence Microscope (TIRFM), Fluorescence-PALM (FPALM), Stochastical Optical Reconstruction Microscopy (STORM), Fluorescence Imaging with One-Nanometer Accuracy (FIONA), and combinations thereof. For examples: Chi, 2009 “Super-resolution microscopy: breaking the limits,” Nature Methods 6(1): 15-18; Blow 2008, “New ways to see a smaller world,” Nature 456:825-828; Hell, et al, 2007, “Far-Field Optical Nanoscopy,” Science 316: 1153; R. Heintzmann and G. Ficz, 2006, “Breaking the resolution limit in light microscopy,” Briefings in Functional Genomics and Proteomics 5(4):289-301; Garini et al., 2005, “From micro to nano: recent advances in high-resolution microscopy,” Current Opinion in Biotechnology 16:3-12; and Bewersdorf et al, 2006, “Comparison of I⁵M and 4Pi-microscopy,” 222(2): 105-117; and Wells, 2004, “Man the Nanoscopes,” JCB 164(3):337-340.

In some embodiments, electron microscopes (EM) are used for imaging.

In some embodiments, an imaging step detects a target. In some embodiments, an imaging step localizes a target. In some embodiments, an imaging step provides three-dimensional spatial information of a target. In some embodiments, an imaging step quantifies a target. By using multiple contacting and imaging steps, provided methods are capable of providing spatial and/or quantitative information for a large number of targets in surprisingly high throughput. For example, when using F detectably different types of labels, spatial and/or quantitative information of up to F_{N} targets can be obtained after N contacting and imaging steps.

Certain techniques for imaging are known in the art. See, for example, International PCT Patent Application No. PCT/US2014/036258, filed Apr. 30, 2014 and titled MULTIPLEX LABELING OF MOLECULES BY SEQUENTIAL HYBRIDIZATION BARCODING, the entire contents of which are herein incorporated by reference in its entirety for all purposes.

In some embodiments, the method comprises analyzing cell size and shape, markers, immunofluorescence measurements, or any combinations thereof.

### Fluorophores

In some embodiments, the method comprises detecting the probes, detectably labelled probes, or oligonucleotides thereof with fluorophores. In some embodiments, the detectably labelled probe comprises a fluorophore.

In some embodiments, the fluorophore is any fluorophore deemed suitable by those of skill in the arts.

In some embodiments, the fluorophores include but are not limited to fluorescein, rhodamine, Alexa Fluors, DyLight fluors, ATTO Dyes, or any analogs or derivatives thereof. In certain embodiments, the detectable moieties include but are not limited to fluorescein and chemical derivatives of fluorescein; Eosin; Carboxyfluorescein; Fluorescein isothiocyanate (FITC); Fluorescein amidite (FAM); Erythrosine; Rose Bengal; fluorescein secreted from the bacterium Pseudomonas aeruginosa; Methylene blue; Laser dyes; Rhodamine dyes (e.g., Rhodamine, Rhodamine 6G, Rhodamine B, Rhodamine 123, Auramine O, Sulforhodamine 101, Sulforhodamine B, and Texas Red).

In some embodiments, the fluorophores include but are not limited to ATTO dyes; Acridine dyes (e.g., Acridine orange, Acridine yellow); Alexa Fluor; 7-Amino actinomycin D; 8-Anilinonaphthalene-1-sulfonate; Auramine-rhodamine stain; Benzanthrone; 5,12-Bis(phenylethynyl) naphthacene; 9,10-Bis(phenylethynyl)anthracene; Blacklight paint; Brainbow; Calcein; Carboxyfluorescein; Carboxyfluorescein diacetate succinimidyl ester; Carboxyfluorescein succinimidyl ester; 1-Chloro-9,10-bis(phenylethynyl)anthracene; 2-Chloro-9,10-bis(phenylethynyl)anthracene; 2-Chloro-9,10-diphenylanthracene; Coumarin; Cyanine dyes (e.g., Cyanine such as Cy3 and Cy5, DiOC6, SYBR Green I); DAPI, Dark quencher, DyLight Fluor, Fluo-4, FluoProbes; Fluorone dyes (e.g., Calcein, Carboxyfluorescein, Carboxyfluorescein diacetate succinimidyl ester, Carboxyfluorescein succinimidyl ester, Eosin, Eosin B, Eosin Y, Erythrosine, Fluorescein, Fluorescein isothiocyanate, Fluorescein amidite, Indian yellow, Merbromin); Fluoro-Jade stain; Fura-2; Fura-2-acetoxymethyl ester; Green fluorescent protein, Hoechst stain, Indian yellow, Indo-1, Lucifer yellow, Luciferin, Merocyanine, Optical brightener, Oxazin dyes (e.g., Cresyl violet, Nile blue, Nile red); Perylene; Phenanthridine dyes (Ethidium bromide and Propidium iodide); Phloxine, Phycobilin, Phycoerythrin, Phycoerythrobilin, Pyranine, Rhodamine, Rhodamine 123, Rhodamine 6G, RiboGreen, RoGFP, Rubrene, SYBR Green I, (E)-Stilbene, (Z)-Stilbene, Sulforhodamine 101, Sulforhodamine B, Synapto-pHluorin, Tetraphenyl butadiene, Tetrasodium tris(bathophenanthroline disulfonate) ruthenium(II), Texas Red, TSQ, Umbelliferone, or Yellow fluorescent protein.

In some embodiments, the fluorophores include but are not limited to Alexa Fluor family of fluorescent dyes (Molecular Probes, Oregon). Alexa Fluor dyes are widely used as cell and tissue labels in fluorescence microscopy and cell biology. The excitation and emission spectra of the Alexa Fluor series cover the visible spectrum and extend into the infrared. The individual members of the family are numbered according roughly to their excitation maxima (in nm). Certain Alexa Fluor dyes are synthesized through sulfonation of coumarin, rhodamine, xanthene (such as fluorescein), and cyanine dyes. In some embodiments, sulfonation makes Alexa Fluor dyes negatively charged and hydrophilic. In some embodiments, Alexa Fluor dyes are more stable, brighter, and less pH-sensitive than common dyes (e.g. fluorescein, rhodamine) of comparable excitation and emission, and to some extent the newer cyanine series. Exemplary Alexa Fluor dyes include but are not limited to Alexa-350, Alexa-405, Alexa-430, Alexa-488, Alexa-500, Alexa-514, Alexa-532, Alexa-546, Alexa-555, Alexa-568, Alexa-594, Alexa-610, Alexa-633, Alexa-647, Alexa-660, Alexa-680, Alexa-700, or Alexa-750.

In some embodiments, the fluorophores comprise one or more of the DyLight Fluor family of fluorescent dyes (Dyomics and Thermo Fisher Scientific). Exemplary DyLight Fluor family dyes include but are not limited to DyLight-350, DyLight-405, DyLight-488, DyLight-549, DyLight-594, DyLight-633, DyLight-649, DyLight-680, DyLight-750, or DyLight-800.

In some embodiments, the fluorophore comprises a nanomaterial. In some embodiments, the fluorophore is a nanoparticle. In some embodiments, the fluorophore is or comprises a quantum dot. In some embodiments, the fluorophore is a quantum dot. In some embodiments, the fluorophore comprises a quantum dot. In some embodiments, the fluorophore is or comprises a gold nanoparticle. In some embodiments, the fluorophore is a gold nanoparticle. In some embodiments, the fluorophore comprises a gold nanoparticle.

### Washes

In some embodiments, the method of any of the preceding embodiments, comprises optionally washing the sample after each step. In certain embodiments, the sample is washed with a buffer that removes non-specific hybridization reactions. In certain embodiments, formamide is used in the wash step. In certain embodiments, the wash buffer is stringent. In certain embodiments, the wash buffer comprises 10% formamide, 2×SSC, and 0.1% triton X-100s.

Having described the embodiments in detail, it will be apparent that modifications, variations, and equivalent embodiments are possible without departing the scope of the defined in the appended claims. Furthermore, it should be appreciated that all examples in the present disclosure are provided as non-limiting examples.

The following non-limiting methods and examples are provided to further illustrate the embodiments disclosed herein. It should be appreciated by those of skill in the art that the techniques disclosed in the methods and examples that follow represent approaches that have been found to function well in practice, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the embodiments.

The following non-limiting methods are provided to further illustrate the embodiments of the invention disclosed herein. It should be appreciated by those of skill in the art that the techniques disclosed in the examples that follow represent approaches that have been found to function well in the practice of several embodiments of the invention, and thus be considered to constitute examples of modes for its practice. However, those of skill in the art should, in light of the present disclosure, appreciate that many changes can be made in the specific embodiments that are disclosed and still obtain a like or similar result without departing from the spirit and the scope of the invention.

### EXAMPLES

### Example 1

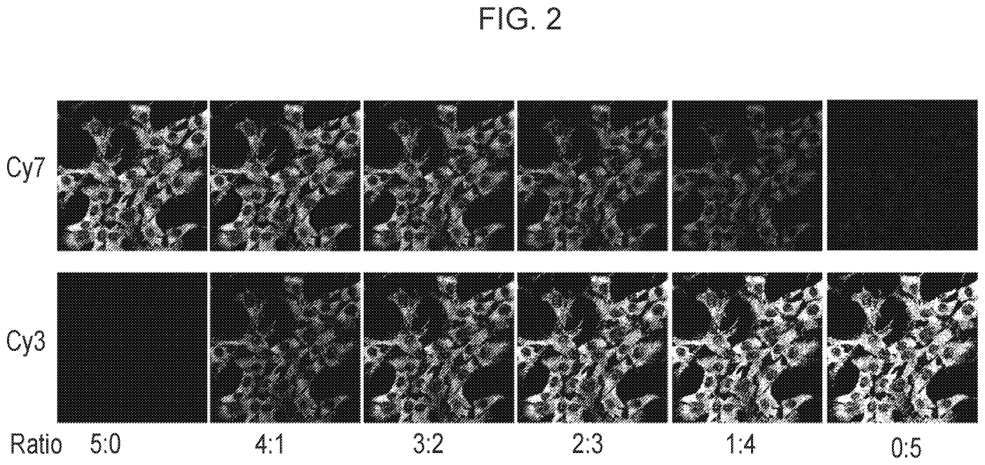

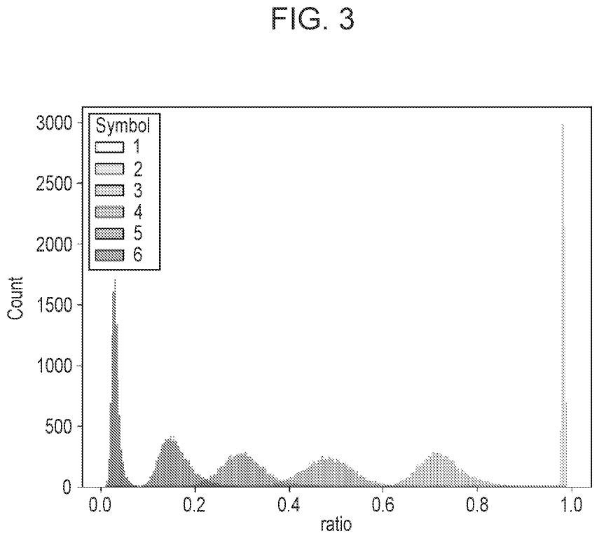

As shown in FIG. 2. Detectably labelled probes were hybridized to a primary probe hybridized to Eukaryotic elongation factor 2 (Eef2) mRNA in mouse NIH/3T3 fibroblast cells and imaged with a confocal microscope.

The Eef2 mRNA was targeted by primary probes, which were amplified through LANTERN. Competitive binding of detectably labelled probes with different ratios generated ratiometric symbols on each RNA molecule.

### Example 2

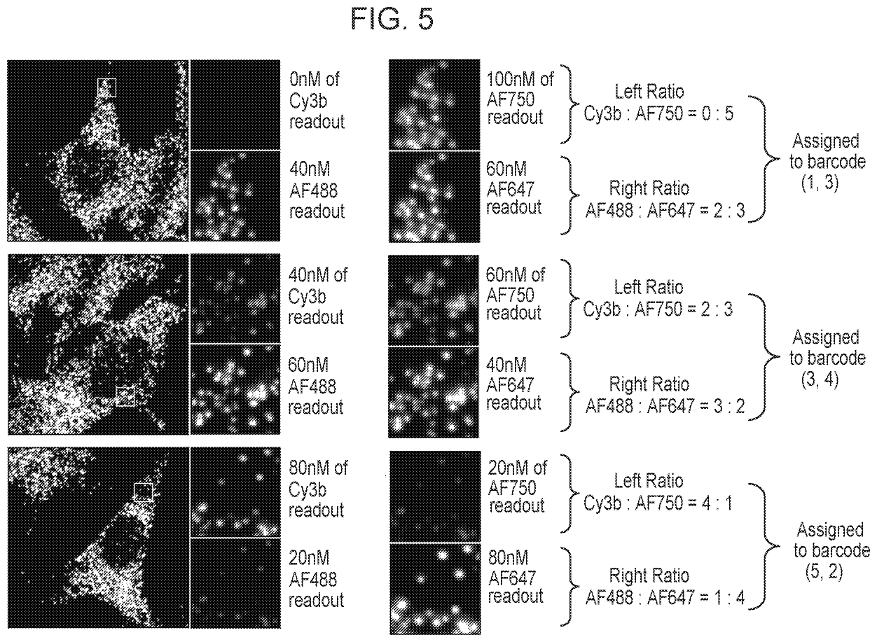

As shown in FIG. 5, ratiometric barcoding was performed in single cells. Thirty six barcodes were demonstrated via a 6×6 ratiometric barcoding scheme.

Eukaryotic elongation factor 2 (Eef2) mRNAs in mouse NIH/3T3 cells were targeted using primary probes with amplifier sequences for LANTERN amplification. The oligonucleotide Sequence of Secondary LANTERN amplifier was:

GAAAGGGTCGAGTTTTTAAAAGGATTCGTGACGGCGACGTTTTGACTTT
AATAAAGGATTCGTGACGGCGACGTTTTGACTTTAAAAGTGCAATGCGA
AC.

The oligonucleotide sequence of the Tertiary LANTERN amplifier was:

CGTCACGAATCCTTTAAAAAACTCGACCCTTTCGTTCGCATTGCACTTT
TTAAAACTCGACCCTTTCGTTCGCATTGCACTTTATAAGTCAAAACGTC
GC.

Both left side and right side adaptors were prepared according to these ratios 0:5, 1:4, 2:3, 3:2, 4:1, 5:0.

The right side adaptors comprised:

Adaptor-1:
GAAAGGGTCGAGTTTAATAGCATCCACTTCCAATCCC
Adaptor-2:
GAAAGGGTCGAGTTTAACACACTTCGCCACTCAGAAC.

The left side adaptors comprised:

Adaptor-1:
CGATAACCTAACCGTGCTGCTTAAGTGCAATGCGAAC
Adaptor-2:
CACTGGTGATAACGCTAACCTTAAGTGCAATGCGAAC.

The adaptor probe mixtures were incubated with their corresponding detectably labelled probes. Cy3 and AF750N were used for the left side detectably labelled probes. Alexa 488 and Alexa 647 were used for the right side detectably labelled probes.

The detectably labelled probes comprised:

/5Alex488N/TGGGATTGGAAGTGGATGCTA
/5Alex647N/GTTCTGAGTGGCGAAGTGTG
/5Alex750N/GGTTAGCGTTATCACCAGTG
/CY3B/GCAGCACGGTTAGGTTATCG.

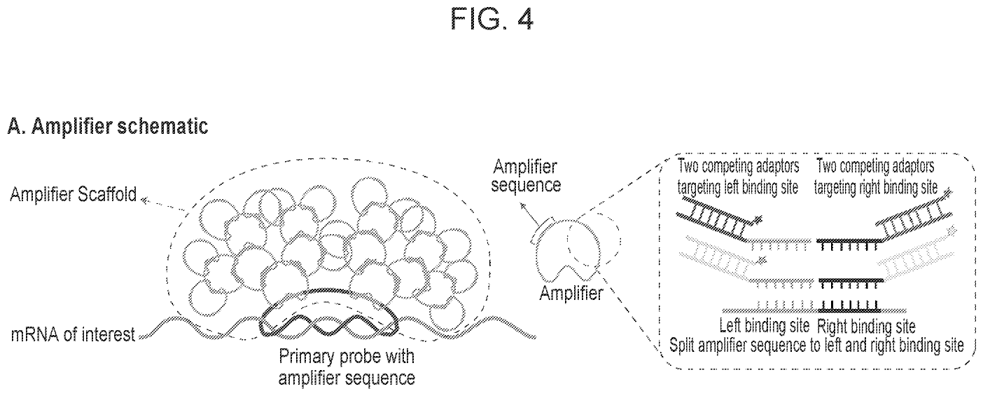

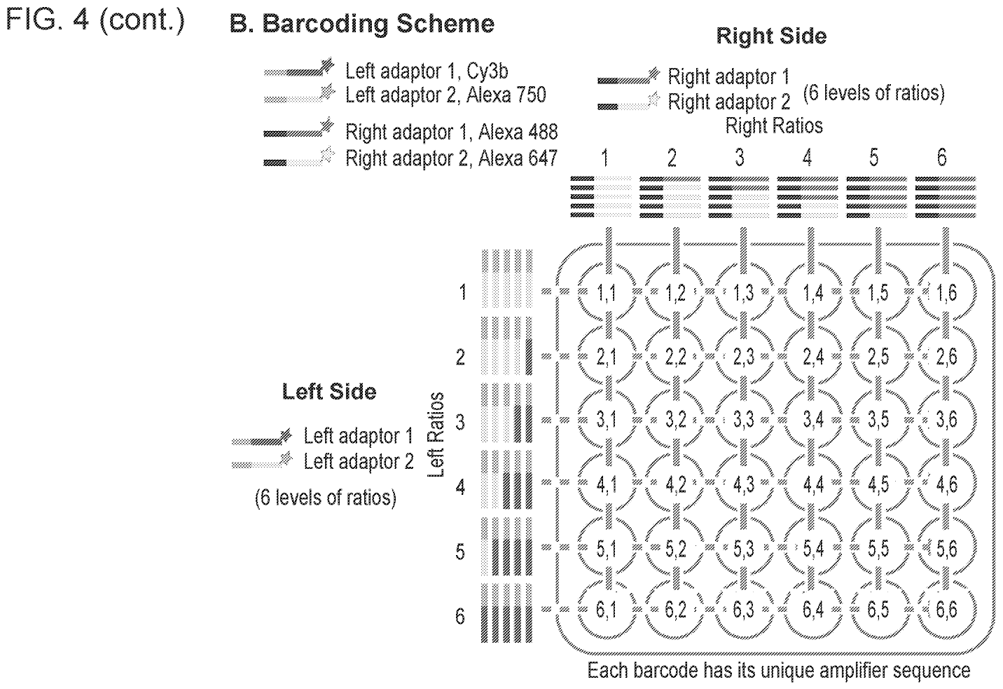

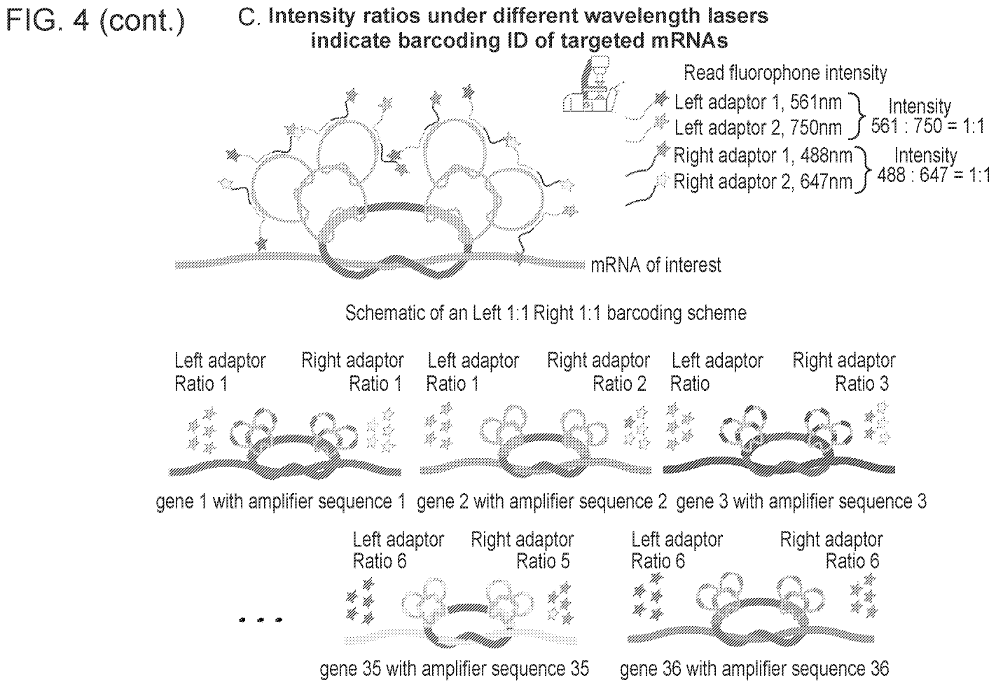

The probes were mixed as shown in FIG. 4B to generate 36 distinct readout mixtures encoding for 36 ratiometric symbols.

The mixtures were then sequentially flowed into the sample via a fluidic system for hybridization. After hybridization, a wash was performed and the sample was imaged by fluorescent microscope using lasers set to wavelengths of 730 nm, 647 nm, 561 nm, 488 nm, and 405 nm.

The left side adaptor ratios were calculated using mRNA dot intensity ratio between the Cy3b and AF750N channels. The right side adaptor ratios were calculated using mRNA dot intensity ratio between the AF488 and AF647 channels. The ratiometric symbols were generated from the left and right ratios.

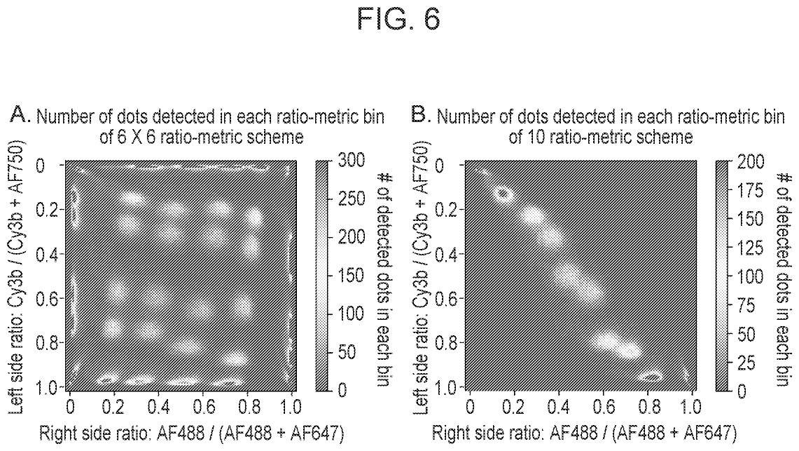

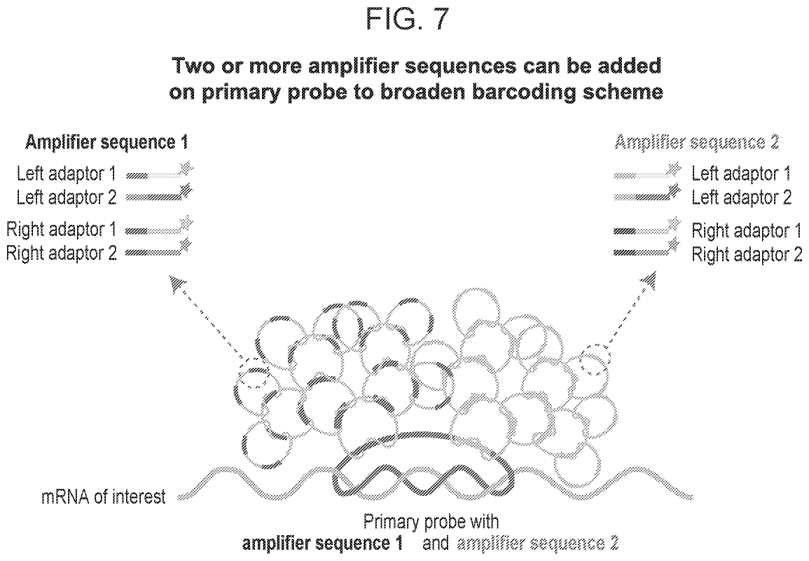

As shown in FIG. 6A, a 2D histogram was constructed that showed that by plotting the 2D histogram of calculated left and right side ratio of all detected dots, 35 clusters can be recovered from 36 barcodes. Further, as shown in FIG. 6B, a 2D heatmap was constructed showing that 10 distinct ratiometric symbols could be generated by using the concentration ratio of 0:9, 1:8, 2:7, 3:6, 4:5, 5:4, 6:3, 7:2, 8:1, 9:0 for both left and right adaptors.

### Example 3

Eukaryotic elongation factor 2 (Eef2) mRNAs in mouse NIH/3T3 cells were targeted using primary probes with amplifier sequences for LANTERN amplification. Three adaptors were designed to interact with detectably labelled probes for three fluorophores AF 647, AF 488, and Cy3b.

The adaptors were competitively mixed to interact with the same oligo binding site on an amplifier sequence.

In this way, an 18 ratiometric symbol scheme was generated by mixing the adaptors and corresponding detectably labelled probes using the relative concentration ratios of: 0:1:4, 0:2:3, 0:3:2, 0:4:1, 0:5:0, 1:0:4, 1:2:2, 1:3:1, 1:4:0, 2:0:3, 2:1:2, 2:3:0, 3:0:2, 3:1:1, 3:2:0, 4:0:1, 4:1:0, 5:0:0.

The ratiometric symbols are calculated by imaging the intensity of detected dots by using lasers of wavelength 647 nm, 488 nm and 561 nm.

The results showed that the ratiometric symbols can be calculated using the intensity of detected dots under the same channel within different hybridization cycles. This result showed that the barcoding scheme can be further broadened to design adaptors to compete for the same amplifier binding site.

### REFERENCES

The following references are incorporated by their entirety.
Murgha, Y., Beliveau, B., Semrau, K., Schwartz, D., Wu, C.-T., Gulari, E., & Rouillard, J.-M. (2015). Combined in vitro transcription and reverse transcription to amplify and label complex synthetic oligonucleotide probe libraries. BioTechniques, 58(6), 301-307.
Murgha, Y. E., Rouillard, J.-M., & Gulari, E. (2014). Methods for the preparation of large quantities of complex single-stranded oligonucleotide libraries. PloS One, 9(4), e94752.
Randolph, J. B., and Waggoner, A. S. Stability, specificity and fluorescence brightness of multiply-labeled fluorescent DNA probes. (1997) Nucleic Acids Res 25(14), 2923-9.
Brumbaugh, J. A., Middendorf, L. R., Grone, D. L., and Ruth, J. L. Continuous, on-line DNA sequencing using oligodeoxynucleotide primers with multiple fluorophores. (1988) Proc Natl Acad Sci USA 85(15), 5610-4.
Hughes, T. R. et al. Expression profiling using microarrays fabricated by an ink-jet oligonucleotide synthesizer. (2001) Nat Biotechnol 19(4), 342-7.
U.S. Ser. No. 10/457,980B2. Multiplex labeling of molecules by sequential hybridization barcoding.
Zhang, Y. (2012). Support Vector Machine Classification Algorithm and Its Application. In: Liu, C., Wang, L., Yang, A. (eds) Information Computing and Applications. ICICA 2012. Communications in Computer and Information Science, vol 308. Springer, Berlin, Heidelberg.
Sahoo G. R et. al. Improving Diagnosis of Cervical Pre-Cancer: Combination of PCA and SVM Applied on Fluorescence Lifetime Images. Photonics 2018, 5, 57.

1. A method for barcoding one or more molecular targets with ratiometric symbols, comprising the steps of:
(i) contacting a sample comprising a plurality of molecular targets with a first plurality of one or more primary probes, wherein the one or more primary probes interact with one or more molecular targets, and wherein each primary probe comprises one or more binding sites for a detectably labelled probe;
(ii) contacting the one or more primary probes with one or more sets of ratiometric detectably labelled probes; wherein each set of the ratiometric detectably labelled probes comprise at least:
a first detectably labelled probe that interacts with a first primary probe binding site; and
a second detectably labelled probe that interacts with the first primary probe binding site;
wherein the label of the first detectably labelled probe is different from the label of the second detectably labelled probe; and wherein the first detectably labelled probe and second detectably labelled probe contact the first primary probe binding site at a pre-determined ratio;
(iii) for each set of ratiometric detectably labelled probes, imaging the intensities of the different detectably labels between different channels to determine a distinct ratio, so that the interaction of the detectably labelled probes with their primary probes is detected;
(iv) generating a ratiometric symbol for each ratio; and
(v) optionally repeating steps (ii)-(iv), each time with one or more sets of detectably labelled probes,
so that one or more molecular targets in the sample are described by a barcode, wherein at least one barcode comprises at least one ratiometric symbol, and wherein at least one molecular target can be differentiated from another molecular target in the sample by a difference in their barcodes.

2. A method barcoding one or more molecular targets with ratiometric symbols, comprising the steps of:
(i) contacting a sample comprising a plurality of molecular targets with a first plurality of one or more primary probes, wherein the one or more primary probes interact with one or more molecular targets, and wherein each primary probe comprises one or more amplifier sequences;
(ii) contacting the one or more primary probes with one or more amplifiers to form one or more amplification scaffolds, wherein the amplifiers comprise one or more amplifier sequences, and wherein the amplifiers sequences comprise one or more adaptor sequences;
(iii) contacting the one or more amplifier scaffolds, with one or more sets of ratiometric adaptor probes; wherein each set of the ratiometric adaptor probes comprise at least:
a first adaptor probe that interacts with a first adaptor sequence on the amplifier scaffold; and
a second adaptor probe that interacts with the first adaptor sequence on the amplifier scaffold;
wherein the first adaptor probe and second adaptor probe contact the first adaptor sequence on the amplifier scaffold at a pre-determined ratio;
(iii) contacting the one or more sets of ratiometric adaptor probes with one or more sets of detectably labelled probes; wherein each set of the detectably labelled probes comprise at least:
a first detectably labelled probe that interacts with a first ratiometric adaptor probe; and
a second detectably labelled probe that interacts with a second ratiometric adaptor probe;
wherein the label of the first detectably labelled probe is different from the label of the second detectably labelled probe;
(iv) for each set of ratiometric adaptor probes, imaging the intensities of the different detectably labels between different channels to determine a distinct ratio, so that the interaction of the adaptor probes with their primary probes is detected;
(v) generating a ratiometric symbol for each ratio; and
(vi) optionally repeating steps (ii)-(v), each time with one or more sets of ratiometric adaptor probes,
so that one or more molecular targets in the sample are described by a barcode, wherein at least one barcode comprises at least one ratiometric symbol, and wherein at least one molecular target can be differentiated from another molecular target in the sample by a difference in their barcodes.

3. The method of any of claim 1 or 2, wherein the sets of detectably labelled probes comprise:
a third detectably labelled probe that interacts with a first primary probe binding site; and
wherein the label of the third detectably labelled probe is different from the label of the first or second detectably labelled probe; and wherein the first detectably labelled probe, second detectably labelled probe, and third detectably labelled probe contact the first primary probe binding site at a pre-determined ratio.

4. The method of claim 3, wherein the sets of ratiometric detectably labelled probes comprise:
a fourth detectably labelled probe that interacts with a first primary probe binding site; and
wherein the label of the fourth detectably labelled probe is different from the label of the first, second, or third detectably labelled probe; and wherein the first detectably labelled probe, second detectably labelled probe, and third detectably labelled probe contact the first primary probe binding site at a pre-determined ratio.

5. The method of claim 1, wherein step (ii) contacts with 2, 3, 4, 5, 6, 7, or 8 sets of ratiometric detectably labelled probes.

6. The method of claim 2, wherein step (iii) contacts with 2, 3, 4, 5, 6, 7, or 8 sets of ratiometric adaptor probes.

7. The method of any of claim 1 or 2, comprising amplifying the primary probes by rolling circle, padlock, branched DNA, ClampFISH, LANTERN, or any combination thereof before step (ii).

8. The method of any of claim 1 or 2, wherein the targets are selected from transcripts, RNA, DNA loci, chromosomes, DNA, proteins, lipids, glycans, cellular target, organelles and any combinations thereof.

9. The method of any of claim 1 or 2, wherein the primary probes are selected from proteins, modified proteins, RNA, oligonucleotides, antibodies, antibody fragments, and combinations thereof.

10. The method of any of claim 1 or 2, wherein each primary probe comprises a nucleic acid sequence complementary to a target nucleic acid sequence.

11. The sequence complementarity of claim 10, wherein the percentage of sequence complementarity is at least 60%, 65%, 70%, 75%, 80%, 85%, 90%, 91%, 92%, 93%, 94%, 95%, 96%, 97%, 98%, 99%, or 100%.

12. The methods of any of claim 1 or 2, wherein the detectably labelled probes are selected from proteins, modified proteins, RNA, oligonucleotides, antibodies, antibody fragments, and combinations thereof.

13. The method of any of claim 1 or 2, wherein each detectably labelled probe comprises a nucleic acid sequence complementary to a primary probe binding site for a detectably labelled probe.

14. The method of claim 13, wherein the percentage of sequence complementarity is at least 60%, 65%, 70%, 75%, 80%, 85%, 90%, 91%, 92%, 93%, 94%, 95%, 96%, 97%, 98%, 99%, or 100%.

15. The method of claim 12, wherein the detectably labelled probes comprise oligonucleotides with the same sequence.

16. The method of claim 12, wherein the detectably labelled probes comprise oligonucleotides with different sequences.

17. The method of any one of the preceding claims, wherein the detectably labelled probes comprise oligonucleotides that are at least 17 nucleotides in length.

18. The method of any one of claims 12-17, wherein the detectably labelled probes interact with the binding sites on the primary probes through one or more intermediate probes.

19. The method of claim 18, wherein the intermediate probes are selected from proteins, modified proteins, RNA, oligonucleotides, antibodies, antibody fragments, and combinations thereof.

20. The method of claim 19, wherein the intermediate probes are oligonucleotides.

21. The method of claim 19, wherein the intermediate probes hybridizes to primary probe.

22. The method of claims 18-21, wherein each intermediate probe comprises a sequence complementary to the primary probe and an overhang sequence.

23. The method of claim 22, wherein the overhang sequence is complementary to a detectably labelled probe.

24. The method of claim 22, wherein the overhang sequence is complementary to a bridge probe.

25. The method of claim 24, wherein the bridge probe is complementary to a detectably labelled probe and to an intermediate probe.

26. The methods of any of claims 1-4, wherein the ratiometric symbols are generated by using different concentrations of detectably labeled probes to compete directly or indirectly for the binding sites on the primary probe.

27. The methods of any of claim 26, wherein the different concentrations of detectably labelled probes are different ratios of detectably labelled probes.

28. The methods of any of claims 1-4, wherein each pre-determined ratio between any two detectably labelled probes is greater to or equal to 0.0.

29. The methods of any of claims 1-4, wherein each pre-determined ratio between any two detectably labelled probes is less than or equal to 1.0.

30. The methods of any of claims 1-4, wherein each pre-determined ratio between any two detectably labelled probes is between about 0.0 to 1.0.

31. The methods of any of claims 1-4, wherein each pre-determined ratio between any two of three detectably labelled probes is between about 0.0 to 1.0.

32. The methods of any of claims 1-4, wherein each pre-determined ratio between any two of four or more three detectably labelled probes is between about 0.0 to 1.0.

33. The methods of any of claims 1-4, wherein each pre-determined ratio between any two of five, six, seven, or eight or more detectably labelled probes is between about 0.0 to 1.0.

34. The method of any of claims 1-4, wherein each pre-determined ratio between any two detectably labelled probes is about 0.1, 0.2, 0.25, 0.3, 0.4, 0.5, 0.6, 0.7, 0.75, 0.8, 0.9, or 1.0.

35. The method of any of claims 1-4, wherein each pre-determined ratio between any three probes is 0.10:0.20:0.70; 0.25:0.25:0.50; 0.25:0.50:0.25; 0.50:0.25:0.25; or 0.70:0.20:0.10.

36. The method of any of claims 1-4, wherein each pre-determined ratio between any four probes is 0.10:0.10:0.10:0.70; 0.10:0.20:0.20:0.50; 0.25:0.25:0.25:0.25; 0.50:0.20:0.20:0.10; 0.70:0.10:0.10:0.10.

37. The method of claim 2, wherein the adaptor probe is an intermediate probe.

38. The method of claim 37, wherein the adaptor probes are selected from proteins, modified proteins, RNA, oligonucleotides, antibodies, antibody fragments, and combinations thereof.

39. The method of claim 38, wherein the adaptor probes are oligonucleotides.

40. The method of claim 18, wherein the adaptor probe hybridizes to an amplifier scaffold.

41. The method of claims 39-40, wherein each adaptor probe comprises a sequence complementary to the primary probe and an overhang sequence.

42. The method of claim 41, wherein the overhang sequence is complementary to a detectably labelled probe.

43. The method of claim 42, wherein the overhang sequence is complementary to a bridge probe.

44. The method of claim 43, wherein the bridge probe is complementary to a detectably labelled probe and to an adaptor probe.

45. The methods of claim 2, wherein the ratiometric symbols are generated by using different concentrations of ratiometric adaptor probes to compete directly or indirectly for the binding sites on the amplifier scaffolds.

46. The methods of any of claim 45, wherein the different concentrations of ratiometric adaptor probes are different ratios.

47. The method of claim 2, wherein each pre-determined ratio between any two ratiometric adaptor probes is greater to or equal to 0.0.

48. The methods of claim 2, wherein each pre-determined ratio between any two ratiometric adaptor probes is less than or equal to 1.0.

49. The methods of claim 2, wherein each pre-determined ratio between any two ratiometric adaptor probes is between about 0.0 to 1.0.

50. The methods of claim 2, wherein each pre-determined ratio between any two of three ratiometric adaptor probes is between about 0.0 to 1.0.

51. The methods of claim 2, wherein each pre-determined ratio between any two of four or more ratiometric adaptor probes is between about 0.0 to 1.0.

52. The methods of claim 2, wherein each pre-determined ratio between any two of five, six, seven, eight, or more ratiometric adaptor probes is between about 0.0 to 1.0.

53. The method of claim 2, wherein each pre-determined ratio between any two ratiometric adaptor probes is about 0.1, 0.2, 0.25, 0.3, 0.4, 0.5, 0.6, 0.7, 0.75, 0.8, 0.9, or 1.0.

54. The method of claim 2, wherein each pre-determined ratio between any three ratiometric adaptor probes is 0.10:0.20:0.70; 0.25:0.25:0.50; 0.25:0.50:0.25; 0.50:0.25:0.25; or 0.70:0.20:0.10.

55. The method claim 2, wherein each pre-determined ratio between any four ratiometric adaptor probes is 0.10:0.10:0.10:0.70; 0.10:0.20:0.20:0.50; 0.25:0.25:0.25:0.25; 0.50:0.20:0.20:0.10; 0.70:0.10:0.10:0.10.

56. The method of claim 1 or 2, further comprising the steps of:
(vi) contacting a sample comprising a plurality of molecular targets with a first plurality of detectably labelled probes comprising at least:
(i) a first detectably labelled probe that interacts with a first molecular target; and
(ii) a second detectably labelled probe that interacts with a second molecular target;
wherein the first detectably labelled probe is different from the second detectably labelled probe;
(vii) imaging the sample after the first contacting step so that interaction of the detectably labelled probes with their target nucleic acids is detected;
(viii) generating a non-ratiometric symbol for each molecular target; and
(ix) repeating the contacting and imaging steps, each time with a new plurality of detectably labelled probes, so that a molecular target in the sample is described by a barcode, wherein at least one barcode comprises at least one non-ratiometric symbol and at least one ratiometric symbol, and wherein the barcode can be differentiated from another target nucleic acid in the sample by a difference in their barcodes.

57. The method of claim 56, wherein the non-ratiometric symbols are generated before the ratiometric symbols are generated.

60. The method of claim 56 wherein the non-ratiometric symbols are generated during generation of the ratiometric symbols.

61. The method of claim 56, wherein the non-ratiometric symbols are generated after the ratiometric symbols are generated.

62. The method of any of the previous claims, wherein the barcode comprises ratiometric, non-ratiometric symbols, and any combination thereof.

63. The method of any of claim 62, wherein the barcode comprises 1, 2, 3, 4, 5, 6, 7, 8, 9, or 10 ratiometric symbols.

64. The method of claim 62, wherein the barcode comprises 0, 1, 2, 3, 4, 5, 6, 7, 8, 9, or 10 non-ratiometric symbols.

65. The method of any of the preceding claims, wherein the sample is washed after each step.

66. The method of claim 65, wherein the sample is washed with a buffer that removes non-specific hybridization reactions.

67. The method of any of claims 1-4, wherein the method further comprises an error correction step.

68. The method of claim 67, wherein the error correction step comprises performing additional rounds of contacting and imaging prior or in between or after steps (i)-(v).

69. The method of claim 1, wherein the assignment of ratiometric symbols comprises applying a machine learning algorithm.
